(19) United States
(12) Patent Application Publication
(10) Pub. No.: US 2024/0192220 A1
(43) Pub. Date: Jun. 13, 2024

(54) Carbon Nanohorns/Nafion/Fe3O4@Pd immunosensor for Shrimp Tropomyosin

(71) Applicant: Universiti Brunei Darussalam, Bandar Seri Begawan (BN)

(72) Inventors: Nura Fazira NOOR AZAM, Bandar Seri Begawan (BN); Minhaz Uddin AHMED, Bandar Seri Begawan (BN); Noor Faizah MOHD-NAIM, Bandar Seri Begawan (BN)

(21) Appl. No.: 18/583,498

(22) Filed: Feb. 21, 2024

Related U.S. Application Data
(62) Division of application No. 17/377,389, filed on Jul. 16, 2021.

(51) Int. Cl. G01N 33/58; B82Y 5/00; B82Y 25/00; B82Y 30/00; B82Y 35/00; B82Y 40/00; C01G 55/00; G01N 27/30; G01N 27/327; G01N 33/12; G01N 33/543; G01N 33/547; G01N 33/68; H01F 1/06

(52) U.S. Cl. CPC G01N 33/587; C01G 55/004; G01N 27/308; G01N 27/3278; G01N 33/12; G01N 33/54366; G01N 33/547; G01N 33/582; G01N 33/68; H01F 1/068; B82Y 5/00; B82Y 25/00; B82Y 30/00; B82Y 35/00; B82Y 40/00; C01P 2004/64; C01P 2006/42

(57) ABSTRACT

The present application discloses an electrochemiluminescence immunosensor. The immunosensor includes an electrode functionalized by a nanocomposite film. The film further includes carbon nanohorns dispersed in Nafion® perfluorinated resin solution. The polymeric solution is further stabilized by magnetic nanoparticles. The immunosensor is a Point of care (POC)-based. The immunosensor is configured to work in the range from 100 ng/mL to 1 fg/mL, and has tendency to detect even traces of the tropomyosin. The immunosensor is capable to detect traces even less than 1 fg/mL, hence having high specificity for Tro-Ag detection in food products with distinguished repeatability.

### TECHNICAL FIELD

The present application relates to detection of food allergens. In particular, the present application relates to an electrochemiluminescence immunosensor for the detection of trace amount of tropomyosin, and a method thereof. The present invention also relates to a nanocomposite for the detection of the tropomyosin.

### BACKGROUND

Food allergy is defined by an abnormal immune response after eating a particular food, and is one of the global health concerns for both children and adults. Ingestion of such allergens may instigate mild and acute symptoms such as diarrhea, nausea, and anaphylaxis. One of the most prevalent food allergens present in marine food diet such as shrimps, shellfish, crabs, oyster, squid, and other invertebrates is tropomyosin. Tropomyosin is a two-stranded alpha-helical, thin filament protein found in cytoskeletons and the tropomyosin found in marine food diet is the heat-stable food allergens. As the allergenicity due to such allergen varies in different individuals, there is no immunosensor developed yet to detect such allergen. Furthermore, even a trace amount of such allergen can be harmful if consumed by those allergic to it. Hence, it is very important to monitor such allergens.

There are many point-of-care (POC) biosensors available which can monitor food allergens. The biosensors are defined as analytical devices which are found to be highly sensitive in detecting foodborne-pathogens and allergens. Such biosensors can similarly detect food allergens. There are different types of biosensors depending on the requirements, for example enzymatic, DNA-based, and immunosensors. There are many conventional methods for the detection of food allergens such as radioallergosorbent test (RAST), enzyme allergosorbent test (EAST), rocket immunoelectrophoresis (RIE), enzyme-linked immunosorbent assay (ELISA), dot immunoblotting, protein chip, etc. However, as biosensors have high selectivity and rapid in obtaining results, they are preferred over the conventional methods. There are some biosensors developed for the detection of tropomyosin, for example electrochemical immunosensor, fluorescent aptasensor, etc. The electrochemical immunosensor includes magnetic beads functionalized with carboxyl groups and customized magnetic nanoparticles on a screen-printed carbon electrode. The fluorescent aptasensor includes magnetic aptamer-immobilized detection probe. However, these strategies have a very low sensitivity of detecting tropomyosin.

Therefore, there exists a need for developing biosensor or immunosensor that have higher sensitivity of detecting even trace amounts of tropomyosin.

### SUMMARY

In a first aspect, the present application discloses a nanocomposite film. The film includes carbon nanohorns (CNHs-OH); Nafion® perfluorinated resin solution; and magnetic nanoparticles. The magnetic nanoparticles are iron oxide supported by palladium-based nanoparticles. The film further includes at least 0.1 mg/ml of the carbon nanohorns; and at least 0.1% of each of the Nafion® perfluorinated resin solution, and magnetic nanoparticles. The carbon nanohorns are dispersed in the Nafion® perfluorinated resin solution, thereby getting oxidized. An antibody may be entrapped on the film via electrostatic interaction and physical adsorption.

In a second aspect, the present application discloses an electrochemiluminescence immunosensor. The immunosensor includes an electrode functionalized by a nanocomposite film. The film further includes carbon nanohorns dispersed in Nafion® perfluorinated resin solution. The polymeric solution is further stabilized by magnetic nanoparticles. The immunosensor includes at least 0.1 mg/ml of the oxidized carbon nanohorns; and at least 0.1% of iron oxide-palladium nanoparticles being immobilized on the SPE. In some embodiments, the immunosensor further includes measuring electrical signal through a [Ru(bpy)³]²⁺/TPrA electrochemiluminescence system. The system has [Ru(bpy)³]²⁺ as a luminophore and Tripropylamine (TPrA) as a co-reactant on an interface between the nanocomposite film and the modified electrode. A redox reaction of electron transfer takes place between the modified electrode's surface and [Ru(bpy)³]²⁺/TPrA ECL system.

The immunosensor is a point-of-care (POC)-based device. The immunosensor is configured to work in the range of 100 ng/ml to 1 fg/mL, and has tendency to detect even traces amount of the tropomyosin. The immunosensor is capable of detecting traces even less than 1 fg/mL, hence having high specificity for Tro-Ag detection in food products with distinguished repeatability.

In yet another aspect, the present application discloses a method for detecting an analyte in a food sample. The method involves fabricating an immunosensor. The fabrication further involves a number of steps, sequence thereof may be exemplary for the skilled persons to understand the present application. The fabrication firstly includes preparing at least 0.1 mg/mL of an oxidized solution of carbon nanohorns. The oxidized solution can be prepared by dispersing the carbon nanohorns in at least 0.1% of Nafion® perfluorinated resin solution. The fabrication involves synthesizing magnetic nanoparticles simultaneously through another method involving a number of steps sequence thereof again may be exemplary for the skilled persons to understand the present application. The method involves initial mixing of at least 4 mL of ultrapure water and at least 10 mM of ascorbic acid, followed by adding at least 10 mM of K₂PdCl₆ thereto. At least 4 mL of 0.1% of magnetic nanoparticles such as Fe₃O₄ are dispersed in ultrapure water. The method further includes stirring the above solution at 700 rpm for at least 1 hour at a temperature of 60° C. Thereafter, magnetic separation is performed for at least 3 minutes and washing thereof with ultrapure water, preparing the magnetic iron oxide-palladium nanoparticles. The method includes further redispersing the magnetic nanoparticles in at least 2 mL of the ultrapure water.

Finally, the fabrication method involves combining the oxidized solution of carbon nanohorns with the iron oxide-palladium nanoparticles, followed by stirring for at least 3 hours at 60° C., synthesizing a nanocomposite film. The method involves dropping at least 3 μL of the synthesized nanocomposite film onto a screen-printed electrode until completely drying, fabricating the immunosensor. Thereafter, at least 3 μL of the food sample is loaded onto the immunosensor, followed by incubating for at least 30 minutes, forming an immunocomplex between a binding agent on the immunosensor and the sample. The electrode undergoes washing with at least 10 m-M of Phosphate-buffered saline (PBS) buffer at pH 7.4, removing unreacted proteins from the sample. An electrical signal may be detected on the electrode, thereby detecting the analyte concentration. In some embodiments, the analyte is a tropomyosin, and the binding agent is an antibody. The electrode is a carbon screen-printed electrode.

### BRIEF DESCRIPTION OF DRAWINGS

The accompanying figures (FIGS.) illustrate embodiments and serve to explain principles of the disclosed embodiments. It is to be understood, however, that these figures are presented for purposes of illustration only, and not for defining limits of relevant applications

FIG. 1 shows a schematic representation of a modified carbon SPE with CNHs-OH/Nafion® perfluorinated resin solution/Fe₃O₄@Pd and the signal produced in the (a) absence and (b) presence of Tro-Ag;

FIG. 2A shows a bar diagram representing the ECL intensity for layer-by-layer characterization; FIG. 2B shows a plot between current and voltage for the redox probe; FIG. 2C shows a chronocoulometry (CC) graph obtained for (i) bare, (ii) nanocomposite film (OH/Nafion® perfluorinated resin solution/Fe₃O₄@Pd)-modified electrode, (iii) modified electrode in the absence of Tro-Ag and (iv) in the presence of 100 pg/mL Tro-Ag; and FIG. 2D shows a bar graph representation of the data taken from FIG. 2C for easier visualization;

FIG. 3A shows a calibration curve of the ECL intensity measured with the immunosensor for the determination of different concentrations of Tro-Ag ranging from 1 fg/mL to 10 pg/mL (104 fg/mL); FIG. 3B shows a comparison of ECL responses of separate immunosensors incubated with tropomyosin, BSA, casein, lysozyme, and ovalbumin, respectively; FIG. 3C shows a graph elucidating the ECL responses recorded with five separate immunosensors; and FIG. 3D shows ECL intensity of different electrodes spiked with 100 pg/mL Tro-Ag on different days (6th, 9th, 12th, 15th, and 18th) after the fabrication day;

FIG. 4A shows the ECL signal of 100 μM luminol in 10 mM PBS buffer, pH 7.4; FIG. 4B shows the ECL signal of 1 mg/mL of CdTeQDs in a mixture of 1 mM TPrA and 10 mM PBS, pH 7.4; and FIG. 4C shows the ECL signal of 800 μM [Ru(bpy)³]²⁺ and 20 mM TPrA in 10 mM PBS, pH 7.4;

FIG. 5 shows the ECL response of different molar ratio of [Ru(bpy)³]²⁺ to TPrA;

FIG. 6 shows a bar graph representing the ECL response from the electrode with three different concentrations of anti-Tro (0.1, 1.0, and 10 μg/mL) in the absence (i) and presence (ii) of 100 pg/mL Tro-Ag;

FIG. 7A shows ECL signal for different incubation time of anti-Tro in the [Ru(bpy)³]²⁺/TPrA ECL system in 10 mM PBS, pH 7.4; FIG. 7B shows ECL signal with four blocking time of 15, 30, 45, and 60 min, and FIG. 7C shows ECL signal with different Ab−Ag reaction time, in the absence (i) and presence (ii) of 100 pg/mL Tro-Ag;

FIG. 8 shows the ECL intensity of [Ru(bpy)³]²⁺/TPrA obtained at various pH, ranging from 5.4 to 9.4;

FIG. 9 shows the measured ECL intensity for layer-by-layer characterization of (i) bare, (ii) nanocomposite-modified electrode, (iii) modified electrode in the absence of Tro-Ag, and (iv) in the presence of 100 pg/mL Tro-Ag;

FIG. 10 shows Nyquist plots illustrating the data obtained with electrochemical impedance spectroscopy (EIS) technique for layer-by-layer characterization of (i) bare, (ii) nanocomposite-modified electrode, (iii) modified electrode in the absence of Tro-Ag, and (iv) in the presence of 100 pg/mL Tro-Ag;

FIG. 11A shows Field Emission—Scanning Electron Microscopy (FE-SEM) image of bare carbon screen-printed electrode (SPE); FIG. 11B shows Field Emission—Scanning Electron Microscopy (FE-SEM) image of CNHs-OH/Nafion® perfluorinated resin solution/Fe₃O₄@Pd-modified SPE; FIG. 11C shows Field Emission—Scanning Electron Microscopy (FE-SEM) image of CNHs-OH/Fe₃O₄@Pd-modified SPE; FIG. 11D shows Field Emission—Scanning Electron Microscopy (FE-SEM) image of Fe₃O₄@Pd-modified SPE; FIG. 11E shows Field Emission—Scanning Electron Microscopy (FE-SEM) image of CNHs-OH/Nafion® perfluorinated resin solution-modified SPE; FIG. 11F shows Field Emission—Scanning Electron Microscopy (FE-SEM) image of CNHs-OH modified SPE.

FIG. 12A shows a calibration curve of the ECL intensity measured with CNHs-OH/Nafion/Fe₃O₄@Pd-modified SPE for the determination of different concentration of Tro-Ag ranging from 10 pg/mL to 100 ng/ml; while FIG. 12B shows the calibration curve for the concentration ranging from 1 fg/mL to 100 ng/ml; and

FIG. 13 shows a graph denoting the ECL intensity recorded for (i) 1 fg/mL, (ii) 100 fg/mL, (iii) 1 pg/mL (103 fg/mL), (iv) 10 pg/mL (104 fg/mL), (v) 1 ng/ml (106 fg/mL), (vi) 10 ng/mL (107 fg/mL), and (vii) 100 ng/ml (108 fg/mL), of Tro-Ag; and

FIG. 14 shows a flowchart depicting a method for detecting analyte in a food sample.

FIG. 15 enlists comparison of conventional detection strategies with that of the present embodiment for tropomyosin; and

FIG. 16 enlists real sample analyses with commercially available shrimp and prawn crackers using the present embodiment.

### DETAILED DESCRIPTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

The present application discloses a nanocomposite film which can be utilized as a carrier of antibodies to bind with Tropomyosin in a food sample. Tropomyosin is a heat stable antigen found in food and is considered an allergen. It can be found in marine staple diet such as shrimp, oyster, crabs, shell fish, and other invertebrates. If tropomyosin is consumed by children and/or adults that are allergic to it thereto can cause diarrhea, nausea, etc. The film includes carbon nanohorns (CNHs-OH); Nafion® perfluorinated resin solution; and magnetic nanoparticles. The magnetic nanoparticles are iron oxide supported by palladium-based nanoparticles. The film further includes at least 0.1 mg/mL of the carbon nanohorns; and at least 0.1% of each of the Nafion® perfluorinated resin solution, and magnetic nanoparticles. The carbon nanohorns are dispersed in the Nafion® perfluorinated resin solution, thereby getting oxidized. An antibody may be entrapped on the film via electrostatic interaction and physical adsorption.

In a second aspect, the present application discloses an electrochemiluminescence immunosensor. The immunosensor includes an electrode functionalized by the nanocomposite film. The immunosensor includes at least 0.1 mg/mL of the oxidized carbon nanohorns; and at least 0.1% of iron oxide-palladium nanoparticles being immobilized on the electrode. The electrode is a carbon-printed screen electrode. In some embodiments, the immunosensor further includes measuring electrical signal through a [Ru(bpy)³]²⁺/TPrA electrochemiluminescence system. The system has [Ru(bpy)³]²⁺ as a luminophore and Tripropylamine (TPrA) as a co-reactant on an interface between the nanocomposite film and the modified electrode. A redox reaction of electron transfer takes place between the modified electrode's surface and [Ru(bpy)³]²⁺/TPrA ECL system.

The immunosensor is a Point-of-care (POC)-based device. The immunosensor is configured to work in the range of 100 ng/mL to 1 fg/mL, and has tendency to detect even traces of the tropomyosin. The immunosensor is capable to detect traces even less than 1 fg/mL, hence having high specificity for Tro-Ag detection in food products with distinguished repeatability.

### Experiment—Reagents and Materials

Rabbit polyclonal anti-tropomyosin (anti-Tro) and natural tropomyosin purified from Carolina shrimp (Tro-Ag) were obtained from Indoor Biotechnologies, Inc. (Bangalore, India). Both anti-Tro and Tro-Ag were further diluted in a 10-mM PBS (pH 7.4) and stored at −20° C. Iron oxide water dispersion (Fe₃O₄), 99.5+%, 15-20 nm, and 20% weight in H₂O, was obtained from US Research Nanomaterials, Inc. (TX, USA). A 10-nm Life Science Gold Colloid was purchased from the BBI™ Solutions (Crumlin, UK). Bovine serum albumin (BSA, 96-99%), L-ascorbic acid (AA, ACS reagent, 99%), magnesium chloride hexahydrate (MgCl₂·6H₂O, ACS reagent, 99.0-102.0%), Nafion® perfluorinated resin solution, oxidized carbon nanohorns (CNHs-OH), potassium chloride (KCl), potassium dihydrogen phosphate (KH₂PO₄), potassium ferrocyanide, potassium ferricyanide, potassium hexachloropalladate(IV) (K₂PdCl₆, 99%), sodium azide (NaN₃), sodium chloride (NaCl), sodium phosphate dibasic (Na₂HPO₄, for molecular biology, 98.0%), tris(2,2′-bipyridyl)dichlororuthenium(II) hexahydrate ([Ru(bpy)₃]Cl₂·6H₂O), tripropylamine (TPrA) with 98% purity, and tris hydrochloride (Tris-HCl) were bought from Sigma-Aldrich Co. (Saint Louis, USA). A 10-mM phosphate-buffered saline (PBS) of pH 7.4 was prepared by dissolving KCl, NaCl, KH₂PO₄, and Na₂ HPO₄ in double distilled water. [Ru(bpy)₃]Cl₂·6H₂O and TPrA were also dissolved in double distilled water. Meanwhile, binding buffer (pH 7.2) was prepared by mixing 10-mM Tris-HCl, 150-mM NaCl, 10-mM KCl, and 2.5-mM MgCl and made up to the desired volume with double distilled water. It is contemplated that Nafion® referred hereinafter relates to Nafion® perfluorinated resin solution and has been purchased for experimental purposes.

### Preparation of the Nanocomposite Film

Firstly, a 0.1 mg/mL of oxidized solution of carbon nanohorns (CNHs-OH) is prepared. Such a solution was prepared by dispersing the carbon nanohorns in 0.1% Nafion® and the mixture was sonicated for 1 hour. Simultaneously, the Fe₃O₄@Pd core-shell nanoparticles are synthesized. The synthesis involved initial mixture of 4-mL ultrapure H₂O and 200-μL ascorbic acid [10 mM], followed by adding 200-μL K₂PdCl₆ [10 mM]. Thereafter, 4 mL of 0.1% of Fe₃O₄ is dispersed in ultrapure H2O and stirred at 700 rpm for 1 h (60° C.). The nanoparticles further underwent magnetic separation for 3 min and underwent washing with ultrapure water three times. The nanoparticles were redispersed in 2 mL of ultrapure H₂O. Thereafter, at least 0.1 mg/mL of CNHs-OH in 0.1% Nafion® was combined with the Fe₃O₄@Pd nanoparticles, in a 1:1 volume ratio in separate glass vials. The mixture (nanocomposite film) was then stirred for 3 h at 60° C. and stored at 4° C. for further use.

### Fabrication of Immunosensor

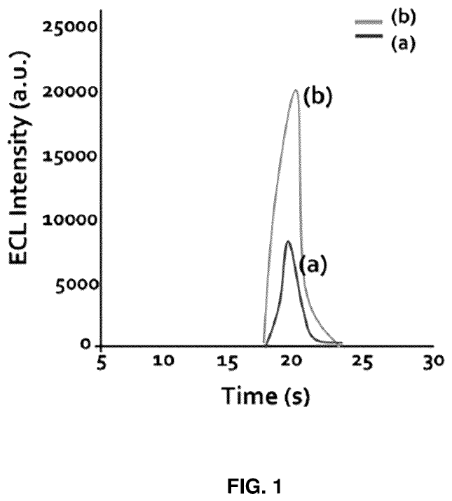

The immunosensor was fabricated by firstly dropping 3 μL of the synthesized the nanocomposite film onto the carbon working electrode of the SPE until it is completely dried. Then, 3 μL of 10 μg/mL anti-Tro was spiked onto the modified working electrode and incubated for 30 min to allow it to be entrapped by the Nafion® film. Afterwards, the electrode's surface was washed with 10-mM PBS (pH 7.4) to remove unbound anti-Tro. Next, 3 μL of 1% BSA dissolved in 0.1% NaN₃ was drop-casted onto the working electrode and left for 45 min. The same buffer (10-mM PBS), pH 7.4 was used for the final washing step, thus completing the preparation of the immunosensor. All of the fabrication processes were performed at room temperature (20° C.±1° C.), in a desiccator. The fabricated immunosensor was then stored at 4° C. until needed. The graphical illustration of the modification on the working electrode of the carbon SPE is shown in FIG. 1.

Electrochemiluminescence (ECL) response of the samples was inspected using BDTeCLP100—an ECL signal recorder, purchased from BioDevice Technology Ltd. (Kanazawa, Japan). The photon counting time was set to 500 m sec, the measurement point was set to 60, a scan rate of 50 mV/s was used, and the potential range was set from 0 to 1.0 V. Disposable screen-printed electrodes (SPE) were acquired from BioDevice Technology Ltd. (Kanazawa, Japan), which consisted of carbon working electrode (with working diameter of 2.64 mm²), counter electrode, and silver reference electrode. Preceding the analyses, 3 μL of the sample was dropped onto the fabricated biosensor and incubated for 30 min, to allow the formation of immunocomplex (between Ab-sample). Subsequently, the electrode was washed with 10-mM PBS buffer, pH 7.4, to remove the unreacted proteins in the sample. ECL measurements were carried out at room temperature (20° C.±1° C.) with 800 μM [Ru(bpy)³]²⁺ and 20 mM TPrA, mixed in 10 mM of PBS buffer (pH 7.4). All of the measurements recorded were obtained at a working potential of 1.0 V.

All of the electrochemical analyses were performed with Autolab PGSTAT101 III (Metrohm, Netherlands) combined with its accompanying software, Nova 1.10. Identically modified SPE chips were utilized as the platform for the electrochemical-based detections. All analyses were performed at room temperature of 20° C.±1° C., and a 5-mM [Fe(CN)⁶]³—/[Fe(CN)⁶]⁴⁻ prepared in 10-mM PBS, pH 7.4, was used as the redox mediator for electrochemical studies. Each of the analyses was replicated three times.

Imitation crab stick, oyster sauce, and rice crackers were purchased from a local store. The food extracts were then prepared according to the manual accompanying the Allergen Extraction kit purchased from Neogen® (USA), with slight modification. Firstly, the extraction solution that consisted of 10-mM PBS of pH 7.4 (included in the kit) was prepared. The extraction solution was pre-heated by immersing the bottle containing the solution in a water bath at 60° C. A 5 g of finely crushed/chopped samples was weighed or 5 mL of liquid sample was placed into a 250-ml bottle. One scoop of extraction powder (included in the kit) was added into the bottle, followed by 125 mL of the pre-heated extraction buffer. The bottle was then sealed tightly to avoid splashing during the extraction process. The bottle was then left shaking at 200 rpm in a water bath at 60° C. for 30 min, after which the bottle was taken out of the water bath and left to stand for 10 min. Finally, 1 mL of the supernatant was pipetted into a fresh, clean microcentrifuge tube and cooled to room temperature prior to analysis. Three microliters of these samples were used for their individual analysis via ECL technique.

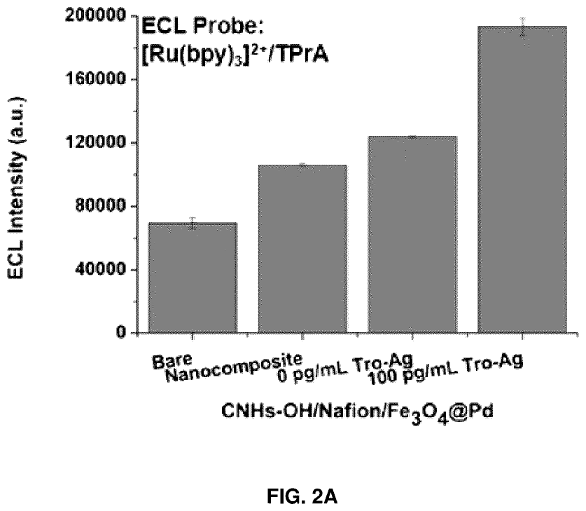

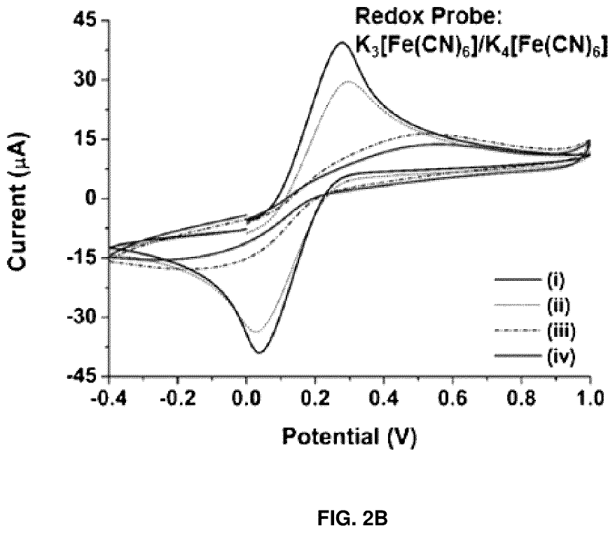

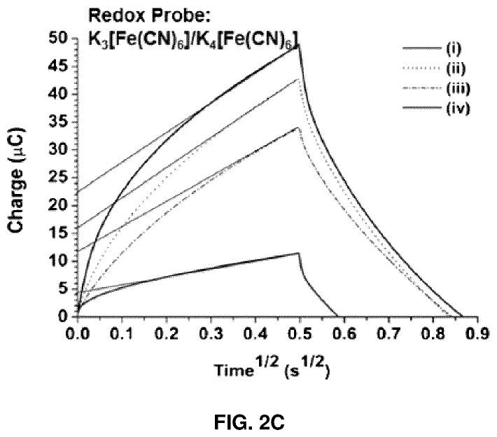

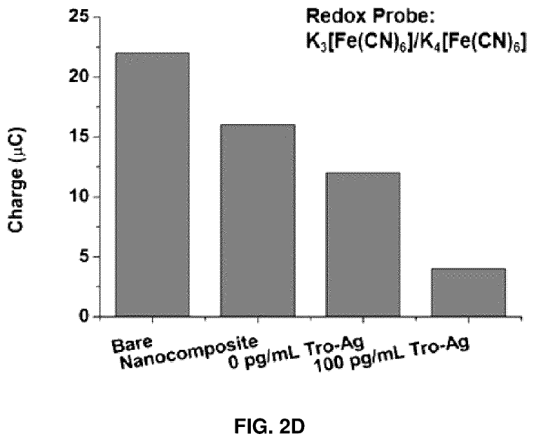

FIG. 2A, FIG. 2B, FIG. 2C, and FIG. 2D show results obtained for electrochemical (ECL) intensity for layer-by layer characterization of the immunosensor when the [Ru(bpy)³]²⁺ is used as a luminophore.

Apart from ECL analyses, cyclic voltammetry (CV) (FIG. 2B), chronocoulometry (CC) (FIG. 2C), and electrical impedance spectroscopy (FIG. 2D), EIS analyses with [Fe(CN)⁶]³⁻/[Fe(CN)⁶]⁴⁻ were also carried out after each fabrication phase to further support the data obtained.

The CV graph after each step of fabrication relied on the redox (reduction-oxidation) reaction of [Fe(CN)⁶]³⁻/[Fe(CN)⁶]⁴⁻. The peak current was markedly reduced as the nanocomposite became completely immobilized onto the surface of the working electrode. Such an immobility may be due to the presence of negatively charged sulfonic acid groups of the Nafion® perfluorinated resin solution, causing a repulsion between the redox species and the electrode's surface. A significant decrease in peak current was observed as the peak current shifted towards the positive potential denoting successful addition of anti-Tro and BSA, which are both negatively charged at pH 7.4, creating stearic hindered environment for rapid electron transfer to take place. After incubation with Tro-Ag, the current decreased further as the Ab−Ag immunocomplexes were formed.

As shown in FIGS. 2C and 2D, the CNHs-OH/Nafion/Fe₃O₄ nanocomposite was productively adsorbed onto the working electrode as the charge was reduced from ˜22 to ˜16 μC, thereby leading to immobilization of the nanocomposite layer and indicated overall negative charge of the nanocomposite was negative, as it comprised Nafion® perfluorinated resin solution and Fe₃O₄@Pd nanoparticles, both of which contained negatively charged groups. As a result, repulsion occurred between the redox probe, i.e. [Fe(CN)⁶]³⁻/[Fe(CN)⁶]⁴⁻ and the nanocomposite, causing a decline in charge measured. Continual reduction was observed after incubation with 1% BSA and 100 pg/mL Tro-Ag, respectively which may be due to the increment of the presence of negatively charged components on the surface of working electrode as both BSA (pI of 5.4) and Tro-Ag (pI of ˜4.6-4.7) are negatively charged at physiological pH 7.4, intensifying the repulsion between the redox probe and the surface of the electrode. The EIS analyses were performed with the frequency range between 100 kHz and 100 MHz and the amplitude of 10 mV.

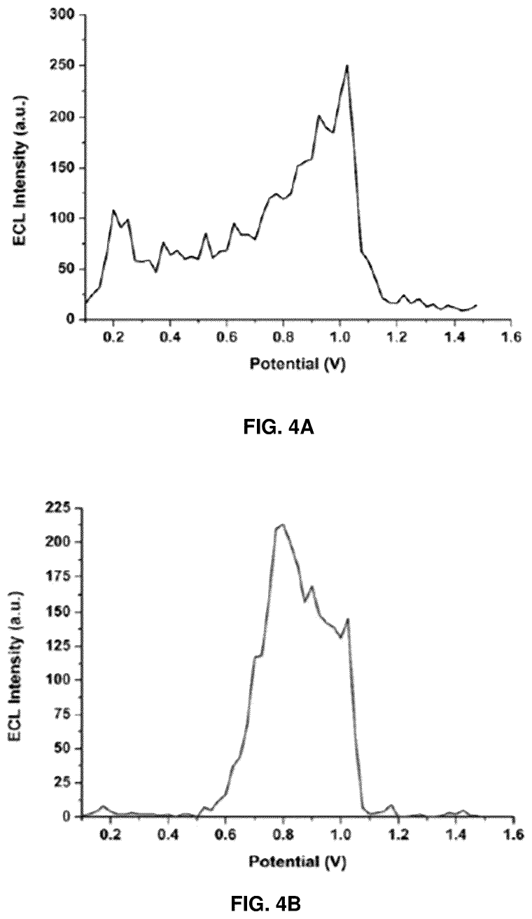

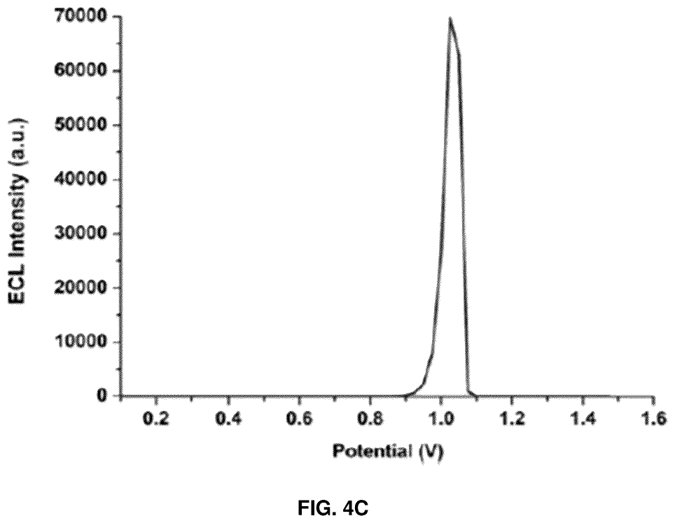

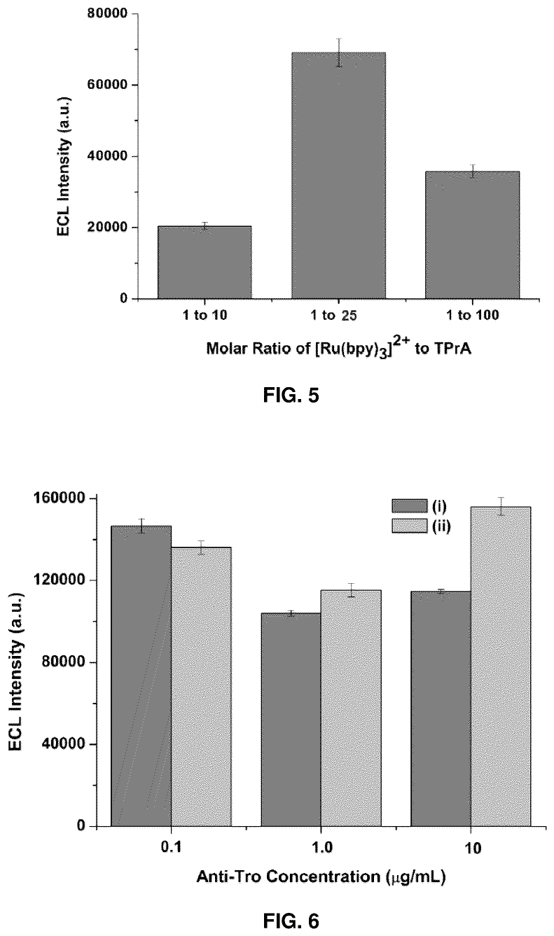

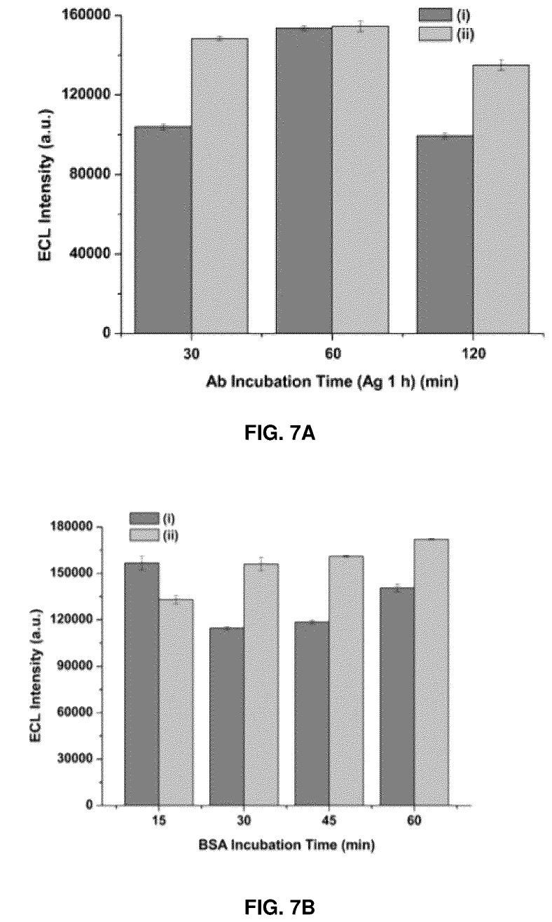

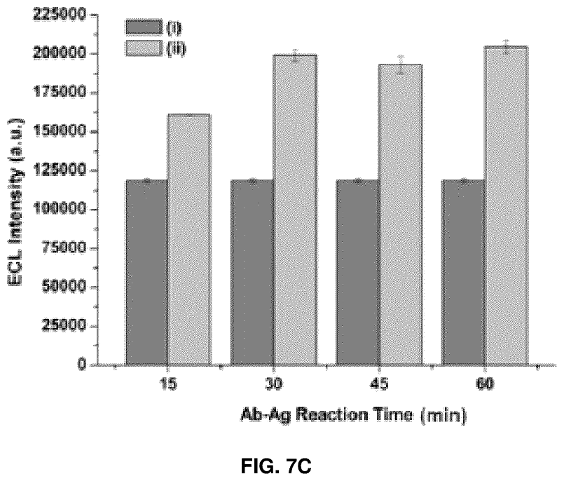

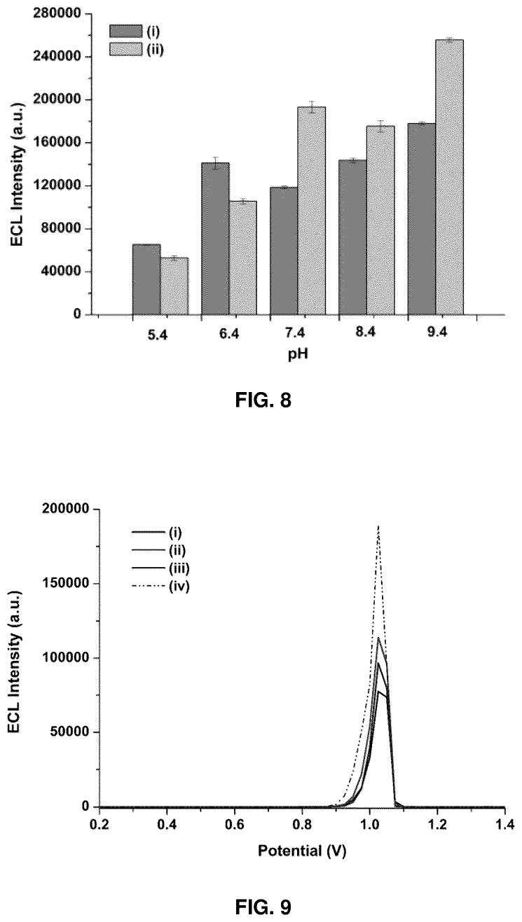

Various ECL luminophore were analyzed for the purpose of finding the optimal luminophore to be used further studies. 100 μM luminol, 1 mg/mL CdTe QDs (cadmium telluride quantum dots), and 800 μM tris(2,2′-bipyridyl)ruthenium(II) ([Ru(bpy)³]²⁺) were investigated as shown in FIG. 4A, FIG. 4B, and FIG. 4C. Luminol and [Ru(bpy)³]²⁺ concentrations were employed, whereas the concentration of CdTe QDs was selected by observing the minimum concentration that can produce ECL signal. All the parameters for recording ECL signals were kept constant for all experiments and performed three times. As shown in FIG. 4A, 800 μM [Ru(bpy)³]²⁺ with TPrA exhibit the highest and smoothest peak compared to the other two luminophores. The ECL peak obtained for luminol and CdTe QDs, respectively, were uneven and the intensity observed was unsatisfactory (FIGS. 4B and 4C). The molar ratio between the luminophore and the coreactant was also optimized as shown in FIG. 5. Various electrode fabrication parameters were additionally examined to establish the ideal conditions for the optimal performance of the immunosensor as shown in FIG. 6. FIG. 7A and FIG. 7B show incubation times of antibodies and blocking agent while the FIG. 7C show reaction-time of Ab−Ag, and the optimal pH (FIG. 8).

The ECL analyses was progressively conducted, starting from the bare carbon SPE, upon addition of nanocomposite, after the immobilization of blocking agent (0 pg/mL Tro-Ag), and finally in the presence of 100 pg/mL Tro-Ag. Immobilization of the CNHs-OH/Nafion/Fe₃O₄@Pd nanocomposite was deduced to be successful as the ECL intensity was improved by ˜1.5 times as shown in FIG. 2A. After incubation with 1% BSA, the ECL signal slightly increased potentially due to the combined effect of the negatively charged BSA (pI=5.4) at pH 7.4 and the presence of the Nation® perfluorinated resin solution, strengthening the attraction towards [Ru(bpy)³]²⁺ instead of blocking the ECL signal (FIG. 9). As more [Ru(bpy)³]²⁺ molecules diffused towards the working electrode's surface and reacted with TPrA, more oxidized species of [Ru(bpy)³]³⁺ were available, which subsequently emitted higher ECL intensity as they returned to their ground state. Finally, after the addition of 100 pg/mL of tropomyosin antigen (Tro-Ag) onto the immunosensor, ECL response was further enhanced. The generation of immunocomplex between anti-Tro and Tro-Ag and the presence of more negatively charged molecules on the working electrode's surface (contributed by Tro-Ag, pI˜4.6-4.7) led to the increased number of [Ru(bpy)³]²⁺ molecules approaching the electrode's surface, resulting in higher ECL signal.

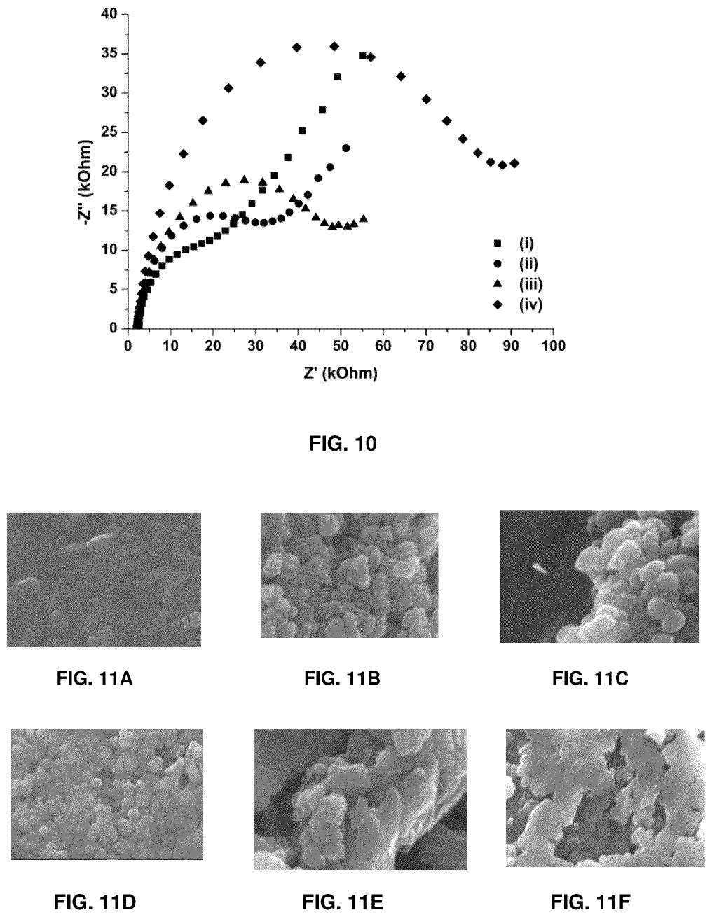

The obtained Nyquist plots (FIG. 10) corroborated the data measured via CV and CC techniques and, thus, confirmed the successful modification on the electrode's surface. Hence, as more layers were added, the electron transfer resistance also increased.

Field emission scanning electron microscopy (FE-SEM) was additionally performed with bare SPE and modified SPEs with different constituents and combinations of the nanocomposite as shown in FIG. 11A, FIG. 11B, FIG. 11C, FIG. 11D, FIG. 11E, and FIG. 11F. FIG. 11A shows Field Emission—Scanning Electron Microscopy (FE-SEM) image of bare carbon screen-printed electrode (SPE). FIG. 11B shows Field Emission—Scanning Electron Microscopy (FE-SEM) image of CNHs-OH/Nafion® perfluorinated resin solution/Fe₃O₄@Pd-modified SPE. FIG. 11C shows Field Emission—Scanning Electron Microscopy (FE-SEM) image of CNHs-OH/Fe₃O₄@Pd-modified SPE. FIG. 11D shows Field Emission—Scanning Electron Microscopy (FE-SEM) image of Fe₃O₄@Pd-modified SPE. FIG. 11E shows Field Emission—Scanning Electron Microscopy (FE-SEM) image of CNHs-OH/Nafion® perfluorinated resin solution-modified SPE. FIG. 11F shows Field Emission—Scanning Electron Microscopy (FE-SEM) image of CNHs-OH modified SPE.

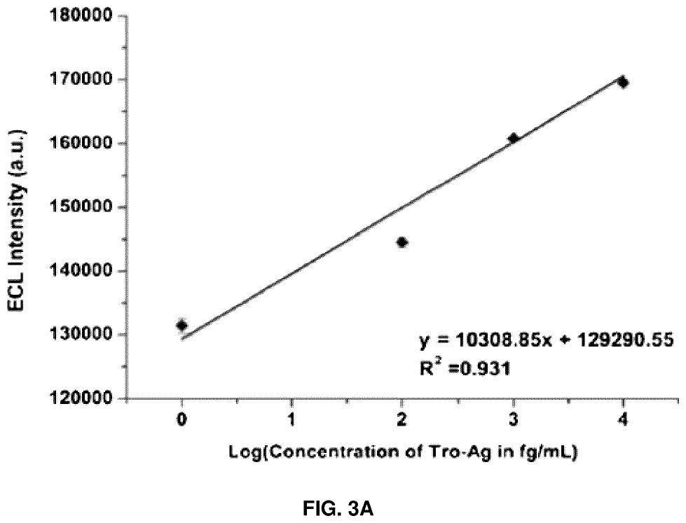

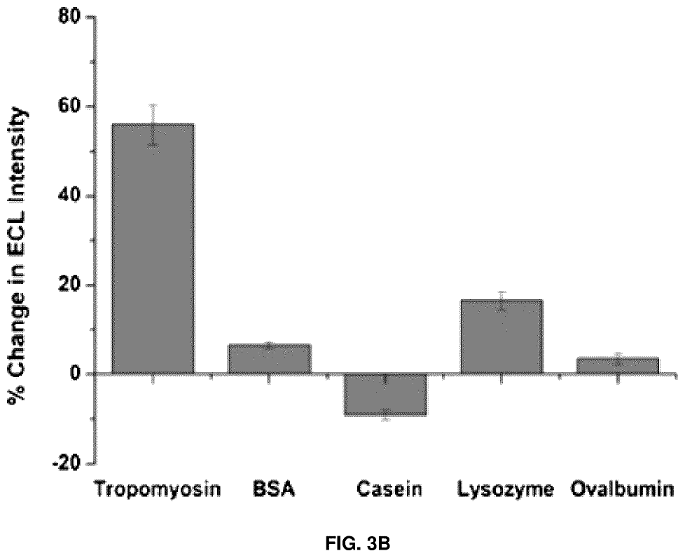

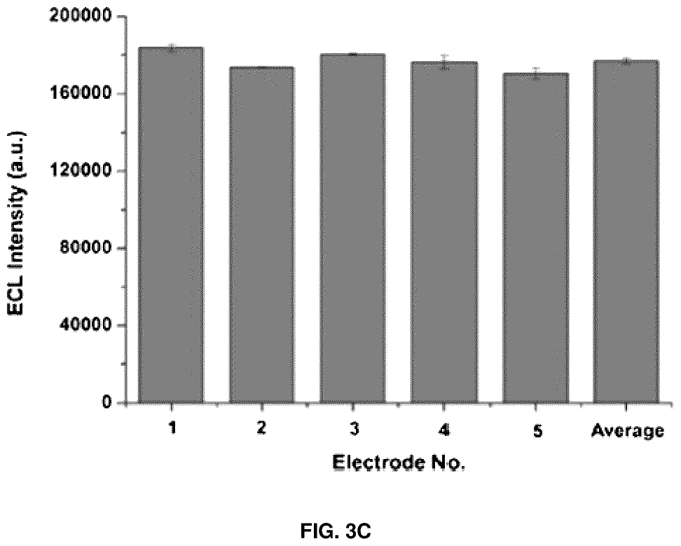

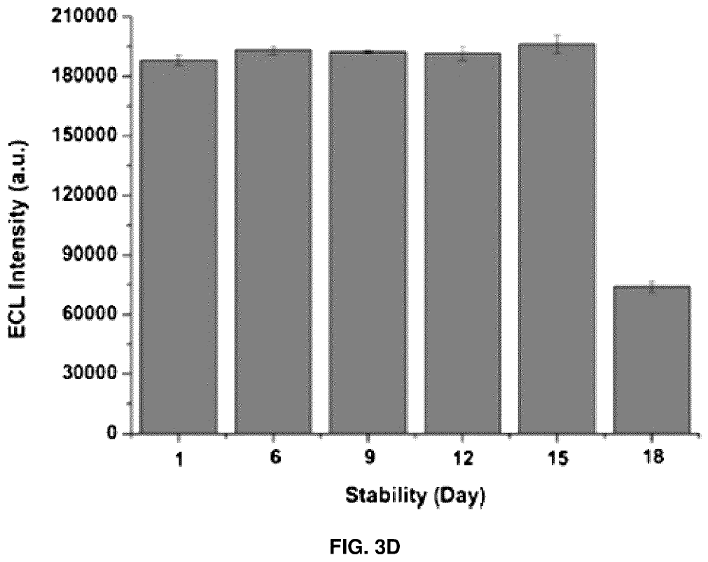

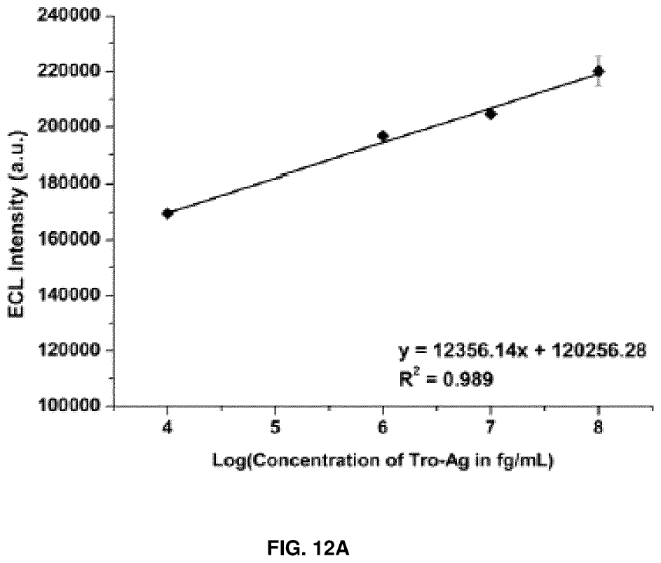

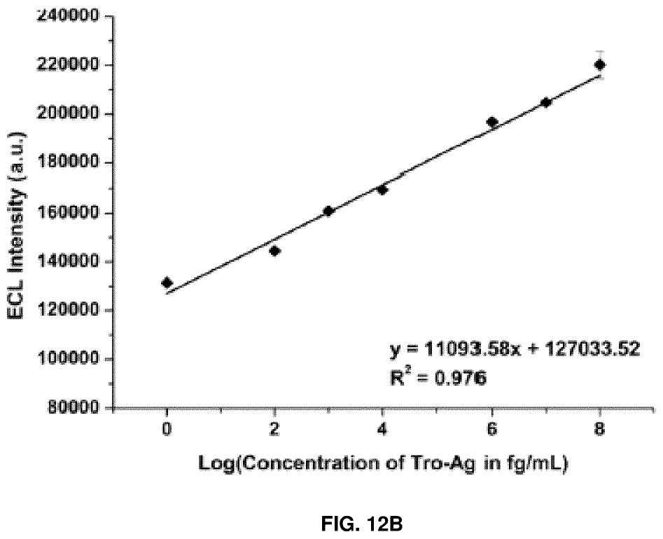

The efficacy of the fabricated ECL immunosensor as a quantitative assay was investigated by varying the concentration of the target antigen (Tro-Ag) from 100 ng/ml to 1 fg/mL. These concentrations were selected in order to focus on developing the immunosensor that is able to detect trace levels of tropomyosin. The resulting data were plotted into two separate graphs. As the concentration of Tro-Ag increased from 1 fg/mL to 10 pg/mL (FIG. 3A) and from 10 pg/mL to 100 ng/ml (FIG. 12A), the ECL response intensified accordingly with a correlation coefficient of 0.931 and 0.989, respectively. The sensitivity of the immunosensor was experimentally and visually deduced via the ECL technique to be 1 fg/mL, which was equivalent to the amount of Tro-Ag. The limit of detection (LOD) can be calculated by using the formula 3 σ/m, where σ is the standard deviation of the y-intercept and m is the gradient of the standard curve. The calculated LOD of our immunosensor was 28.16 fg/mL. The ability to detect trace level of targeted analyte and to function over a wide detection range is advantageous as the minimum concentration of allergen that could trigger an allergic reaction could vary from an individual to another, depending on their susceptibility towards the allergen.

The selectivity of our bioassay was further validated using raw and processed food samples, which typically constituted of numerous proteins and other components that might interfere with the signal production and therefore result in false positive or false-negative results. For this purpose, five different allergen proteins (antigens) at concentration of 100 pg/mL were selected, spiked, and incubated under optimal conditions onto the developed immunosensor. The selected allergens included tropomyosin (Tro), bovine serum albumin (BSA), casein, lysozyme (Lyso), and ovalbumin (OVA) as they are some of the common constituents of processed food products. The observed ECL signals were recorded and expressed as a bar graph in FIG. 3B. From the graph, lysozyme unfavourably generated a modest increase in ECL signal of ˜17%, giving a false-positive reading with the fabricated ECL immunosensor due to polyclonal tropomyosin antibodies that may bind to multiple antigens, compromising the selectivity and specificity of the assay. Lysozyme antigens may have interacted with the incorporated anti-Tro and, ultimately, interfered with the true ECL response. Moreover, with a pI of 11.1, lysozyme is positively charged at pH 7.4 and its conformational stability is dependent on pH when the system pH is less than that of the pI. Therefore, some of lysozyme proteins may tend to agglomerate and remain on the biosensor. When the luminophore solution is introduced, a steric hindrance due to the presence of lysozyme proteins caused the luminophore molecules to diffuse closer to the biosensor's surface, emitting higher ECL intensity in comparison with the analysis with other nonspecific proteins.

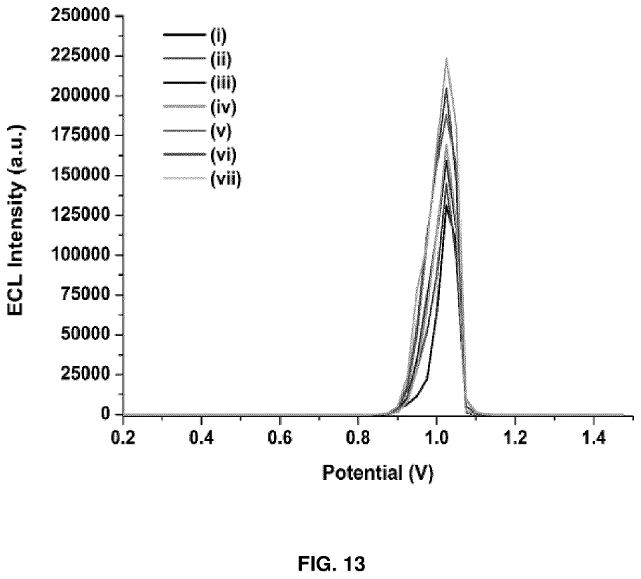

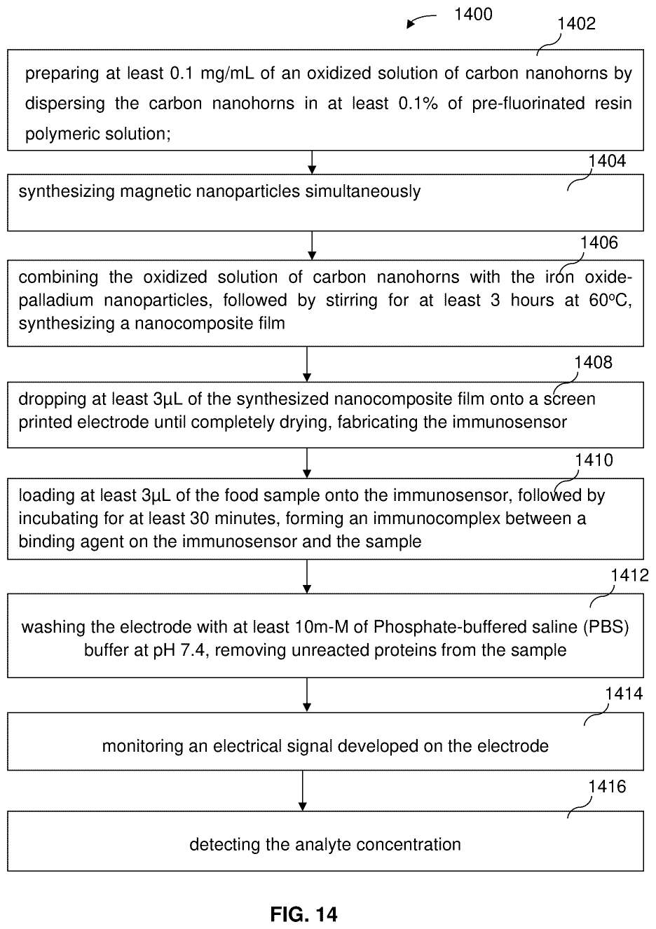

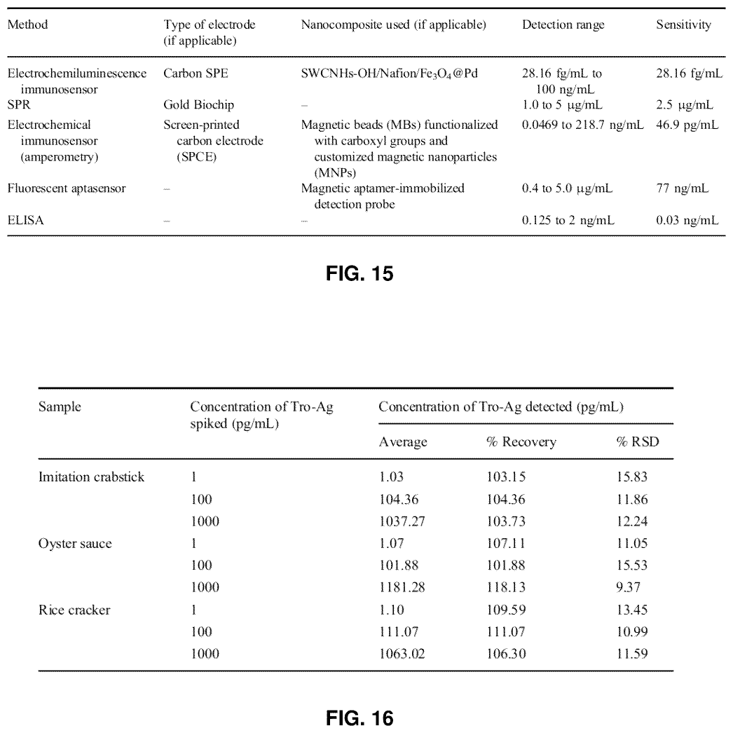

Following this, the repeatability of this developed immunosensor was investigated by incubating 100 pg/mL Tro-Ag with five stand-alone fabricated electrodes (FIG. 3C). The observed ECL signals were then analysed and calculated for their relative standard deviation (% RSD), and the % RSD was established to be 0.811%. Hence, the immunosensor was capable of replicating the results with exceptional precision. The stability of this immunosensor was also examined by incubating 100 pg/mL Tro-Ag with prepared electrodes and analyzing them individually on the 6th, 9th, 12th, 15th, and 18th day after the preparation day (FIG. 3D). A 59.81% decrease in ECL intensity was recorded when using the electrode after 18 days of storage. The immunosensor was able to detect the target antigen (Tropomyosin) with very good stability within 15 days after the fabrication. The % RSD of the immunosensors analyzed over the period of 15 days was calculated to be 1.52%. As shown in FIG. 15, the immunosensor is capable of detecting the target allergen at a wider range as compared to the conventional strategies. The susceptibility of the immunosensor of detecting tropomyosin was evaluated by artificially spiking a known trace concentration of tropomyosin (1, 100, and 1000 pg/mL) into food extracts. The dilution factors used were 1:106, 0.01, 0.10, and 1.0 ng/mL Tro-Ag in 10-mM PBS buffer of pH 7.4. The samples were then incubated onto separate immunosensors at room temperature and the ECL intensity was observed. Respective signals were then analysed and calculated for their respective % recoveries, using the equation obtained from the calibration curve (FIG. 12B), as shown in FIG. 16. FIG. 13 shows a graph denoting the ECL intensity recorded for (i) 1 fg/mL, (ii) 100 fg/mL, (iii) 1 pg/mL (103 fg/mL), (iv) 10 pg/mL (104 fg/mL), (v) 1 ng/ml (106 fg/mL), (vi) 10 ng/ml (107 fg/mL), and (vii) 100 ng/ml (108 fg/mL), of Tro-Ag.

The present application discloses a method 1400 for detecting analyte in a food sample. The method 1400 involves a number of steps, sequence thereof may be exemplary to understand the skilled persons in the art. The method 1400 involves preparing at least 0.1 mg/mL of an oxidized solution of carbon nanohorns by dispersing the carbon nanohorns in at least 0.1% of Nafion® perfluorinated resin solution at step 1402. Thereafter, the method 1400 involves synthesizing magnetic nanoparticles simultaneously at step 1404. The method 1400 involves combining the oxidized solution of carbon nanohorns with the iron oxide-palladium nanoparticles, followed by stirring for at least 3 hours at 60° C., synthesizing a nanocomposite film at step 1406. Thereafter, the method 1400 involves dropping at least 3 μL of the synthesized nanocomposite film onto a screen-printed electrode until completely drying, fabricating the immunosensor at step 1408. At step 1410, at least 3 μL of the food sample is loaded onto the immunosensor, followed by incubating for at least 30 minutes, forming an immunocomplex between a binding agent on the immunosensor and the sample. The electrode undergoes washing with at least 10 m-M of Phosphate-buffered saline (PBS) buffer at pH 7.4, removing unreacted proteins from the sample at step 1412. The method 1400 involves monitoring an electrical signal developed on the electrode at step 1414, followed by finally detecting the analyte/antigen (Tropomyosin) at step 1416.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. The description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

In the application, unless specified otherwise, the terms “comprising”, “comprise”, and grammatical variants thereof, intended to represent “open” or “inclusive” language such that they include recited elements but also permit inclusion of additional, non-explicitly recited elements.

It will be apparent that various other modifications and adaptations of the application will be apparent to the person skilled in the art after reading the foregoing disclosure without departing from the spirit and scope of the application and it is intended that all such modifications and adaptations come within the scope of the appended claims.

1. A method for detecting an analyte in a food sample, the method comprising:
fabricating an immunosensor by a method further comprising:
preparing at least 0.1 mg/mL of an oxidized solution of carbon nanohorns by dispersing the carbon nanohorns in at least 0.1% of Nafion perfluorinated resin solution;
synthesizing magnetic nanoparticles simultaneously by a method further comprising:
mixing at least 4 mL of ultrapure water and at least 10 mM of ascorbic acid, preparing a mixture;
adding at least 10 mM of K₂PdCl₆ to the mixture;
adding at least 4 mL of 0.1% of Fe₃O₄ nanoparticles dispersed in ultrapure water;
stirring the above solution at 700 rpm for t least 1 hour at a temperature of 60° C., followed by magnetic separation for at least 3 minutes and washing with ultrapure water, preparing the magnetic iron oxide-palladium nanoparticles;
redispersing the magnetic nanoparticles in at least 2 mL of the ultrapure water;
combining the oxidized solution of carbon nanohorns with the iron oxide-palladium nanoparticles, followed by stirring for at least 3 hours at 60° C., synthesizing a nanocomposite film;
dropping at least 3 μL of the synthesized nanocomposite film onto a screen-printed electrode until completely drying, fabricating the immunosensor;
loading at least 3 μL of the food sample onto the immunosensor, followed by incubating for at least 30 minutes, forming an immunocomplex between a binding agent on the immunosensor and the sample;
washing the electrode with at least 10 m-M of Phosphate-buffered saline (PBS) buffer at pH 7.4, removing unreacted proteins from the sample;
monitoring an electrical signal developed on the electrode; and
detecting the analyte concentration.

2. The method of claim 1, wherein the analyte is a tropomyosin.

3. The method of claim 1, wherein the binding agent is an antibody.

4. The method of claim 1, wherein the electrode is a carbon screen-printed electrode.

5. The method of claim 1, further comprising:
measuring electrical signal by a [Ru(bpy)₃]²⁺/TPrA electrochemiluminescence system having [Ru(bpy)₃]²⁺ as a luminophore and Tripropylamine (TPrA) as a co-reactant on an interface between the nanocomposite film and the modified electrode.
